(19) United States
(12) Patent Application Publication
(10) Pub. No.: US 2018/0201920 A1
(43) Pub. Date: Jul. 19, 2018

(54) PHORESIS DEVICE

(71) Applicants: Sharp Kabushiki Kaisha, Sakai City, Osaka (JP); The University of Tokyo, Bunkyo-ku, Tokyo (JP)

(72) Inventors: Satoshi IHIDA, Sakai City (JP); Agnes TIXIER MITA, Bunkyo-ku, Tokyo (JP); Hiroshi TOSHIYOSHI, Bunkyo-ku, Tokyo (JP)

(21) Appl. No.: 15/743,704

(22) PCT Filed: Jul. 13, 2016
(86) PCT No.: PCT/JP2016/070656

§ 371 Date: Jan. 11, 2018

(30) Foreign Application Priority Data
Jul. 15, 2015 (JP) 2015-141625

(51) Int. Cl. C12N 13/00; C12N 15/02; C12M 1/00; G01N 27/447

(52) U.S. Cl. CPC C12N 13/00; G01N 27/44791; C12M 45/07; C12N 15/02

(57) ABSTRACT

A phoresis device for moving a target object by dielectrophoresis is realized with use of a thin film transistor substrate. A phoresis device (1) includes a TFT substrate (10) which supports a muscle cell (T1) and a nerve cell (T2) and which is configured to form an electric field that causes dielectrophoresis. In the phoresis device (1), the muscle cell (T1) and the nerve cell (T2) are moved through application of a voltage to part of a plurality of transistors of the TFT substrate (10).

### TECHNICAL FIELD

The present invention relates to a phoresis device (TFT on glass platform for biological applications).

### BACKGROUND ART

In recent years, there has been actively developed a technique of performing, on a target object (sample) such as a cell, various manipulations such as a manipulation of moving the sample.

For example, Patent Literature 1 discloses a device that can move a sample by electrowetting on dielectric (EWOD).

Patent Literature 2 discloses a device that can move a sample by dielectrophoresis (DEP).

### CITATION LIST

### Patent Literature

[Patent Literature 1]
International Publication No. 03/044556 A2 (Publication Date: Jun. 5, 2003)

[Patent Literature 2]
Specification of U.S. Pat. No. 6,977,033 B2 (Registration Date: Dec. 20, 2005)

### SUMMARY OF INVENTION

### Technical Problem

However, Patent Literatures 1 and 2 neither disclose nor suggest a technical idea of realizing, with use of a thin film transistor (TFT) substrate, a phoresis device for moving a target object by dielectrophoresis.

Therefore, with the inventions disclosed in Patent Literatures 1 and 2, it is not possible to realize, with use of a thin film transistor substrate, a phoresis device for moving a target object by dielectrophoresis.

The present invention has been made in view of the above problem, and an object of the present invention is to realize, with use of a thin film transistor substrate, a phoresis device for moving a target object by dielectrophoresis.

### Solution to Problem

In order to attain the above object, a phoresis device in accordance with an embodiment of the present invention is a phoresis device for moving a target object by dielectrophoresis, the phoresis device including: a thin film transistor substrate which supports the target object and which is configured to form an electric field that causes the dielectrophoresis, the thin film transistor substrate including a plurality of transistors, the target object being moved through application of a voltage to part of the plurality of transistors of the thin film transistor substrate.

### Advantageous Effects of Invention

According to a phoresis device in accordance with an embodiment of the present invention, it is possible to realize, with use of a thin film transistor substrate, a phoresis device for moving a target object by dielectrophoresis.

### BRIEF DESCRIPTION OF DRAWINGS

FIG. 1 shows the configuration of a phoresis device in accordance with Embodiment 1 of the present invention.

FIG. 2 shows the relationship between a TFT substrate in accordance with Embodiment 1 of the present invention and members that constitute a TFT liquid crystal panel for use in a display device.

FIG. 3 shows the configuration of the TFT substrate in accordance with Embodiment 1 of the present invention.

(a) through (c) of FIG. 4 are each a cross-sectional view of the TFT substrate shown in FIG. 3.

FIG. 5 indicates an experimental result of the relationship between the frequency of an AC voltage used for dielectrophoresis and a phoresis speed.

(a) through (e) of FIG. 6 each shows how dielectrophoresis occurs at each frequency in a case where the frequency of the AC voltage is changed. (f) of FIG. 6 is a schematic view of a striped pattern of ITO on an experimental circuit.

(a) and (b) of FIG. 7 each show an experimental example of dielectrophoresis performed in the phoresis device in accordance with Embodiment 1 of the present invention.

(a) and (b) of FIG. 8 each illustrate a result of optical observation of a target object in the phoresis device in accordance with Embodiment 1 of the present invention.

(a) through (c) of FIG. 9 each illustrate a manipulation of the target object, other than dielectrophoresis, which is performed in the phoresis device in accordance with Embodiment 1 of the present invention.

(a) of FIG. 10 shows a state where myeloma cells have not been electroporated. (b) of FIG. 10 shows a state where the myeloma cells have been electroporated.

(a) of FIG. 11 is an enlarged view of the electrode shown in FIG. 10. (b) of FIG. 11 is a cross-sectional view taken along the line D-D′ shown in (a) of FIG. 11. (c) of FIG. 11 is a schematic view showing how electroporation is performed. (d) of FIG. 11 is a schematic view showing how a cell membrane is perforated.

FIG. 12 illustrates a case where some portions of the TFT substrate in accordance with Embodiment 1 of the present invention serve as various sensors.

FIG. 13 illustrates a current path in a case where no cell is placed on the TFT substrate in accordance with Embodiment 1 of the present invention.

FIG. 14 illustrates an example of a current path in a case where a cell is placed on the TFT substrate in accordance with Embodiment 1 of the present invention.

FIG. 15 shows the electrical equivalent circuit of a cell.

(a) and (b) of FIG. 16 each show a result of an experiment conducted in the case where the TFT substrate in accordance with Embodiment 1 of the present invention served as an impedance sensor.

(a) and (b) of FIG. 17 each show a result of an experiment conducted in the case where the TFT substrate in accordance with Embodiment 1 of the present invention served as an ISFET sensor.

(a) of FIG. 18 shows the pulsed voltage waveform generated by an AC power source of the phoresis device in accordance with Embodiment 1 of the present invention. (b) of FIG. 18 shows the voltage waveform of a simulated stimulation generated by a simulating circuit of the phoresis device in accordance with Embodiment 1 of the present invention.

FIG. 19 shows the configuration of a modified example of the TFT substrate in accordance with Embodiment 1 of the present invention.

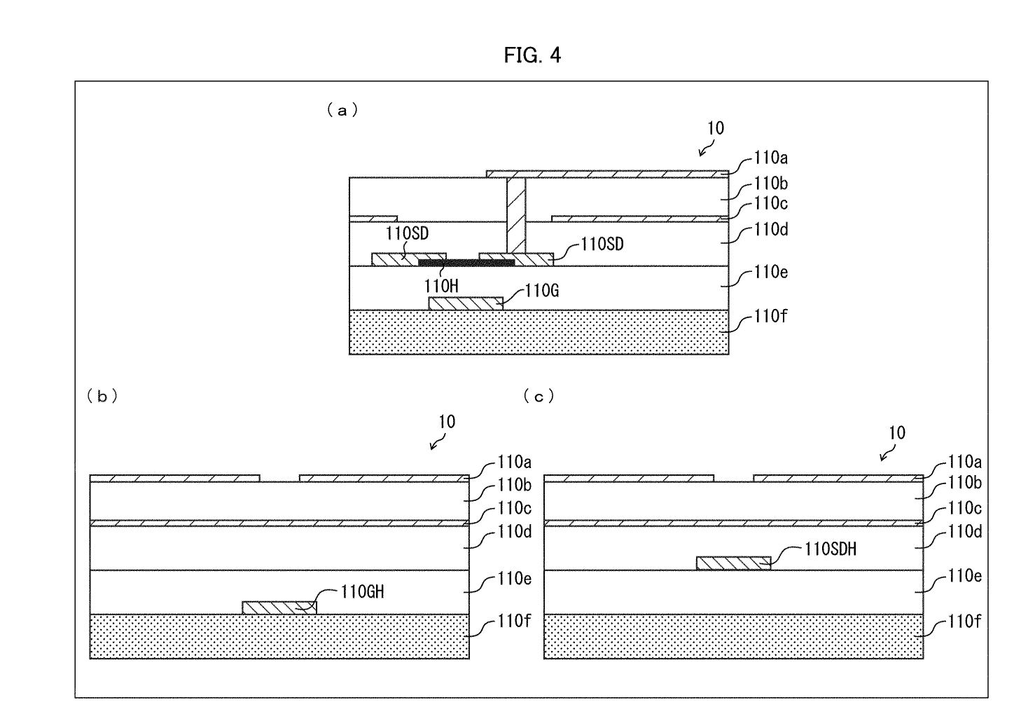

(a) of FIG. 20 shows the positional relationship between pixel electrodes and electrode wirings on the TFT substrate shown in FIG. 3. (b) of FIG. 20 shows the positional relationship between pixel electrodes and electrode wirings arranged on the TFT substrate shown in FIG. 19.

FIG. 21 shows the configuration of a phoresis device in accordance with Embodiment 2 of the present invention.

FIG. 22 shows a modified example of a restricting member of a TFT substrate in accordance with Embodiment 2 of the present invention.

FIG. 23 shows the configuration of an observation system in accordance with Embodiment 3 of the present invention.

### DESCRIPTION OF EMBODIMENTS

### Embodiment 1

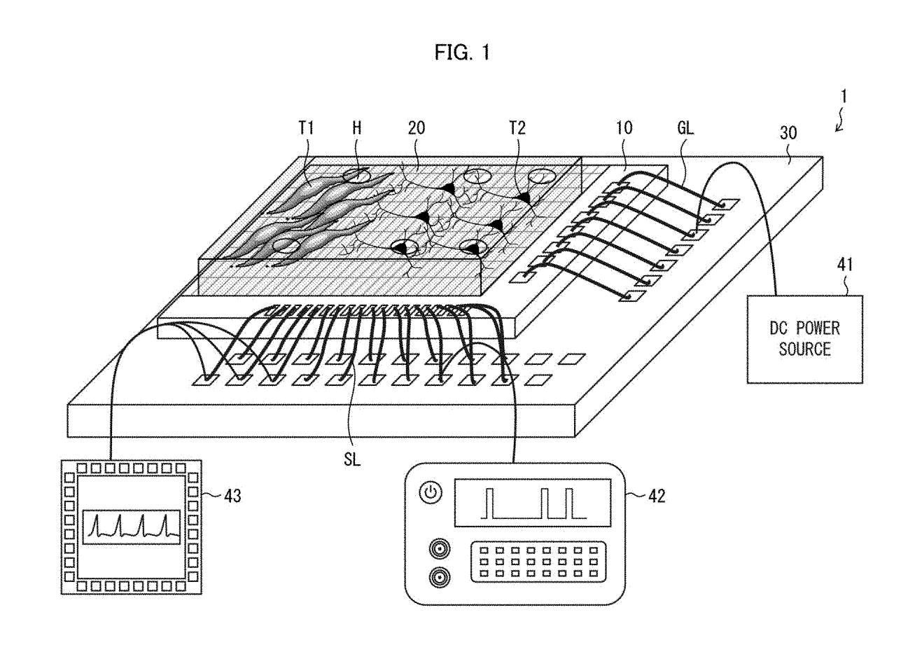

The following description will discuss Embodiment 1 of the present invention with reference to FIGS. 1 through 18.

(Outline of Phoresis Device 1)

FIG. 1 shows the configuration of a phoresis device 1 in accordance with the present embodiment. The phoresis device 1 includes a TFT substrate 10 (thin film transistor substrate), a chamber 20 (restricting member), and a substrate 30.

The phoresis device 1, as described later, serves to move a target object (e.g., a cell) by dielectrophoresis. FIG. 1 shows two kinds of target objects, namely, a muscle cell T1 and a nerve cell T2.

With the phoresis device 1, it is possible to arrange a cell (i.e., the target object) at an intended position by dielectrophoresis. This makes it possible to prevent the cell from adhering to the surface of the TFT substrate 10. Moreover, the phoresis device 1 also makes it possible to perform various manipulations (described later), such as chemical treatment and electrical stimulation, on the cell that has been arranged at the intended position by dielectrophoresis.

In the case of culturing cells on the TFT substrate 10, by performing dielectrophoresis in advance, it is possible to prevent different kinds of cells from being mixed with each other before the cell culturing. This makes it possible to suitably culture cells on the TFT substrate 10.

Moreover, it is possible to use dielectrophoresis to control the direction of the axon of the nerve cell T2 with respect to the muscle cell T1.

The target object of dielectrophoresis is, however, not limited to a cell, and can be any object (e.g., a particle) that can be moved by dielectrophoresis.

(TFT Substrate 10)

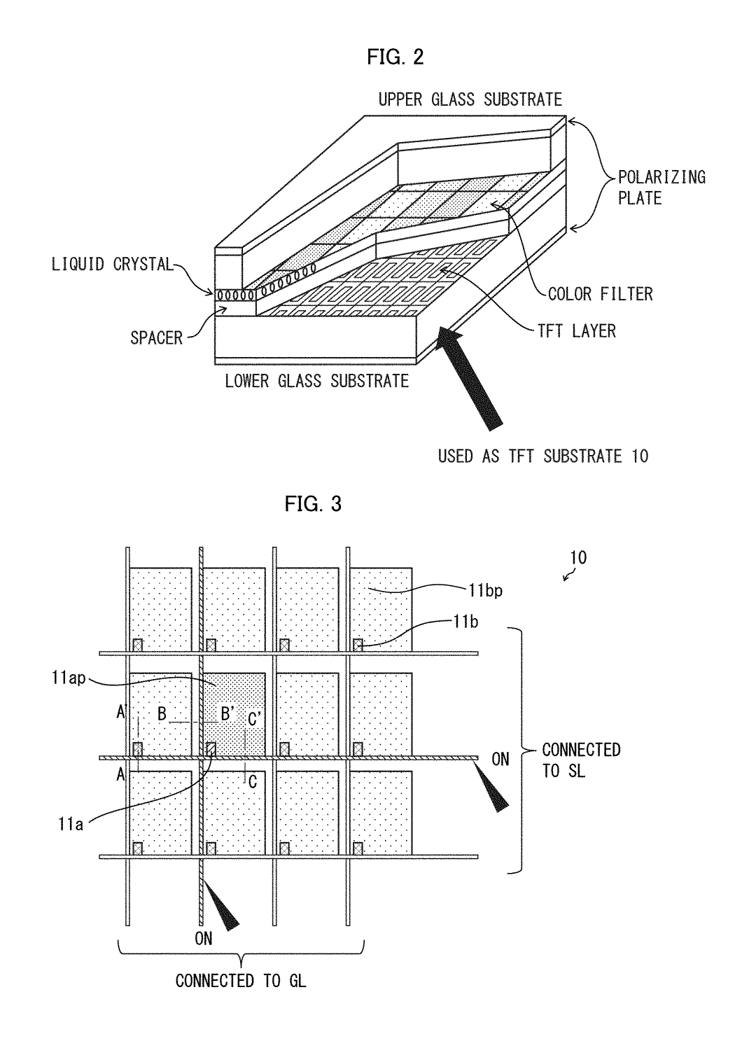

The TFT substrate 10 serves to form an electric field that causes dielectrophoresis by which the target object is moved. The TFT substrate 10 also serves as a supporting member that supports the target object. In the present embodiment, some members of a TFT liquid crystal panel for use in a display device are used as the TFT substrate 10 (see FIG. 2). FIG. 2 shows the relationship between the TFT substrate and the members that constitute the TFT liquid crystal panel for use in a display device.

The TFT liquid crystal panel, as shown in FIG. 2, includes two glass substrates, namely, an upper glass substrate and a lower glass substrate. Note that “upper” refers to a side that faces the viewer who views the display screen of the TFT liquid crystal panel, whereas “lower” refers to a side opposite to the “upper” side.

Between the upper glass substrate and the lower glass substrate, the TFT liquid crystal panel includes polarizing plates, a color filter, a liquid crystal, a spacer, and a TFT layer. Descriptions of these members except for the TFT layer are, however, omitted because they are not related to the present embodiment.

FIG. 3 shows the configuration of the TFT substrate 10. The TFT substrate 10 (more specifically, the TFT layer) includes a plurality of TFTs that are arranged on the TFT substrate 10 in a matrix manner. The TFT layer in accordance with the present embodiment is a member for use in the display screen of a TFT liquid crystal panel and thus has a light-transmitting region. Accordingly, the TFT substrate 10 has a light-transmitting region.

On the TFT substrate 10, the gate electrode of each TFT is connected to a corresponding one of a plurality of first wirings, which are arranged in a first direction so as to be parallel to each other. The first wirings can alternatively be referred to as gate wirings, because they are connected to respective gate lines GL.

Meanwhile, the source electrode of each TFT is connected to a corresponding one of a plurality of second wirings, which are arranged in a second direction so as to be parallel to each other. The second direction can be, for example, a direction perpendicular to the above-described first direction. The second wirings can alternatively be referred to as source wirings, because they are connected to respective source lines SL.

The plurality of TFTs are, as shown in FIG. 3, arranged in a matrix manner so as to be located at the intersections of the gate wirings and the source wirings. This makes it possible to adjust the voltage across the drain electrode of each TFT by adjusting the voltages to be applied to the corresponding gate line GL and source line SL. That is, it is possible to switch each TFT between a conductive state and a non-conductive state (i.e., between an ON-state and an OFF-state).

The drain electrode of each TFT is connected to a corresponding one of pixel electrodes, which are arranged on the TFT substrate 10 in a matrix manner. This makes it possible to switch each of the pixel electrodes, which are connected to the respective drain electrodes, between the ON-state and the OFF-state by adjusting the voltages to be applied to the corresponding gate line GL and source line SL.

For example, in a case where voltages are applied to a single gate line GL and a single source line SL, a TFT 11a (transistor), which is arranged at the intersection between the gate wiring connected to the single gate line GL and the source wiring connected to the single source line SL, is switched to the ON-state (see FIG. 3). Accordingly, it is possible to switch, to the ON-state, a pixel 11ap (in other words, the pixel corresponding to the TFT 11a) that is connected to the drain electrode of the TFT 11a.

In the case of FIG. 3, TFTs other than the TFT 11a are kept in the OFF-state. For example, a TFT 11b that is arranged at a position different from that of the TFT 11a is kept in the OFF-state. Accordingly, the pixel corresponding to the TFT 11b is also kept in the OFF-state.

As such, by switching part of the plurality of TFTs of the TFT substrate 10 to the ON-state, it is possible to form an electric field (i.e., a non-uniform electric field) that causes dielectrophoresis by which the target object placed on the TFT substrate 10 is moved.

Each of the gate wirings and the source wirings of the TFT substrate 10 can be made of, for example, indium tin oxide (ITO). By using ITO, it is possible to make those wirings transparent.

Alternatively, in order to reduce the electrical resistance of the gate wirings and the source wirings, it is possible to use an opaque metal material (e.g., Al) for those wirings. This point will be later described in a modified example. Each of the pixel electrodes can also be made of ITO.

(a) through (c) of FIG. 4 are each a cross-sectional view of the TFT substrate 10 shown in FIG. 3. Specifically, (a) of FIG. 4 is a cross-sectional view taken along the line A-A′ shown in FIG. 3, (b) of FIG. 4 is a cross-sectional view taken along the line B-B′ shown in FIG. 3, and (C) of FIG. 4 is a cross-sectional view taken along the line C-C′ shown in FIG. 3.

The TFT substrate 10, as shown in each of (a) through (c) of FIG. 4, includes ITO electrodes 110a, an interlayer insulating film 110b, COM electrodes 110c, a passivation insulating film 110d, a gate insulating film 110e, a glass substrate 110f, semiconductor layers 110H, source/drain electrodes 110SD, gate electrodes 110G, source/drain wirings 110SDH, and gate electrode wirings 110tH. The configuration shown in FIG. 4, however, will not be described in detail because it is similar to that of a commonly-known TFT substrate for use in a display device.

The COM electrodes 110c each can be grounded so as to stabilize the electric potential difference between the ITO electrodes 110a. The COM electrodes 110c are, however, not essential for the phoresis device 1 to perform dielectrophoresis.

The ITO electrodes 110a each do not have a property that adversely affects a cell which serves as the target object in a case where the cell is brought into contact with any of the ITO electrodes 110a. It is therefore possible to place a cell (and a liquid containing the cell), which is targeted for dielectrophoresis, directly on the surface(s) of the ITO electrode(s) 110a.

In order to prevent the chemical change from occurring on the surfaces of the ITO electrodes when voltages are applied to the ITO electrodes 110a, it is possible to apply an appropriate coating to the surfaces of the ITO electrode 110a. For example, the ITO electrodes 110a can be coated with an insulating film made of a material such as SiO₂, SiNx, or polyimide.

(Chamber 20)

The chamber 20, which is arranged on the TFT substrate 10, is a member that restricts the range of movement of the target object. The chamber 20 is produced preferably as a member having a certain light-transmitting property.

Examples of the material for the chamber 20 include polydimethyl siloxane (PDMS), epoxy resin (particularly, SU-8), polymethyl methacrylate (PMMA), polyvinylidene difluoride (PVDF), glass, and quartz.

By using PDMS as the material for the chamber 20, it is possible to particularly facilitate production of the chamber 20. This is because a chamber 20 made of PDMS can be patterned by mixing two kinds of material liquids, curing the resultant mixture, and shaping (transferring) the cured material on (to) a mold pattern. Therefore, by using PDMS as the material for the chamber 20, it is possible to easily produce the chamber even in a case where the chamber 20 is of a relatively large size.

PDMS is particularly suitable in a case where a cell serves as the target object because, for example, (i) it has excellent adhesion to the substrate, (ii) it has excellent chemical resistance, (iii) it does not emit autofluorescence, and (iv) it has biocompatibility.

Meanwhile, in a case where SU-8 is used as the material for the chamber 20, it is possible to form the chamber 20 by a light exposure process. Therefore, SU-8 is particularly suitable in a case where precise alignment is required for the chamber 20.

The chamber 20 is capable of storing a liquid that contains the target object. The chamber 20 can have, for example, a rectangular shape. In such a case, the chamber 20 can be a frame-like member having no bottom.

In a case where the chamber 20 has a bottom, the chamber 20 can have a space therein for retaining a liquid. In such a case, the chamber 20 can have an injection hole H via which a liquid is injected into the space from the outside.

Provision of the chamber 20 allows the phoresis device to handle a liquid that contains the target object. This is because the chamber 20 makes it possible to prevent the liquid from leaking out of the TFT substrate 10, and in turn makes it possible to prevent the liquid from adversely affecting other members (particularly, electrical members such as the substrate 30).

Provision of the chamber 20 therefore allows the target object contained in the liquid to be more efficiently moved by dielectrophoresis. Provision of the chamber 20 also allows for reduction in strength of the electric field that causes dielectrophoresis. This makes it possible to reduce electrical stress applied to the target object.

The phoresis device 1, however, does not necessarily include the chamber 20. In a case where efficient movement of the target object is not required or where the target object is highly resistant to electrical stress, it is possible to place the target object directly on the surface of the TFT substrate 10 and perform dielectrophoresis on the target object thus directly placed.

Note that, in order to further enhance the efficiency of moving the liquid, it is possible to provide, in the chamber 20 as appropriate, a micro-channel (micro-flow path) via which the flow of a liquid can be controlled. In a case where a plurality of micro-channels are provided, it is also possible to facilitate the manipulation of removing, from the liquid, unnecessary components other than the target object.

(Other Members)

Next, other members of the phoresis device 1 will be described below with reference to FIG. 1. The substrate 30 serves to facilitate electrical connection between the TFT substrate 10 and external devices. However, the phoresis device 1 does not necessarily include the substrate 30, and alternatively, the TFT substrate 10 and the external devices can be directly connected.

In the present embodiment, the substrate 30 is arranged so as to support the TFT substrate 10. Although not shown in the drawings, for example, by hollowing out a portion of the substrate 30 which portion overlaps with the TFT substrate 10, it is possible to observe the TFT substrate 10 from below even in a case where an opaque printed substrate is used as the substrate 30.

A direct-current (DC) power source 41 is, as shown in FIG. 1, connected to the gate lines GL (in other words, gate wirings) via the substrate 30. The DC power source 41 is preferably capable of adjusting the value of a DC voltage which it generates.

Meanwhile, an alternating-current (AC) power source 42 is connected to the source lines SL (in other words, source wirings) via the substrate 30. The AC power source 42 generates a certain AC voltage waveform (e.g., a sinusoidal voltage or a pulse waveform voltage), and is preferably capable of adjusting the frequency and the amplitude of the certain AC voltage waveform. For example, a function generator can be used as the AC power source 42.

The above arrangement makes it possible to (i) apply a DC voltage generated by the DC power source 41 to the gate electrode of a certain TFT and (ii) apply an AC voltage generated by the AC power source 42 to the source electrode of the certain TFT.

A simulating circuit (artificial network) 43 is connected to the source lines SL via the substrate 30. The simulating circuit 43 serves to generate an AC voltage (e.g., the voltage waveform shown in (b) of FIG. 18 described later) that serves as simulated stimulation (artificial stimulation).

The simulating circuit 43 can be realized by, for example, a large scale integrated (LSI) device. The AC voltage generated by the simulating circuit 43 is applied to the source electrode of a certain TFT. Note that the simulated stimulation will be described later in detail.

(Chemical Treatment)

At least the muscle cell T1 or the nerve cell T2 can be subjected to a chemical treatment. The chemical treatment can be a treatment that is performed for the purpose of giving chemical stimulation to the muscle cell T1 or the nerve cell T2.

As described earlier, the chemical treatment can be performed after the muscle cell T1 and the nerve cell T2 are arranged at respective certain positions by dielectrophoresis. Note that a chemical solution with which the chemical treatment is performed can be injected into the chamber 20 via the injection hole H.

For example, in order to give chemical stimulation to the muscle cell T1, it is possible to perform a chemical treatment on the muscle cell T1 with use of GM6, which is a medicine for amyotrophic lateral sclerosis (ALS) manufactured by GENERVON.

Meanwhile, on the nerve cell T2, it is possible to perform a chemical treatment with use of a neuro peptide, which is a chemical substance that stimulates the nerve cell T2.

By performing a chemical treatment CT, it becomes possible to investigate (i) an activated protein C in a specific disease (e.g., ALS) or (ii) other methods of treating a muscle disorder.

(Study on Frequency of AC Voltage Used in Dielectrophoresis)

Prior to an experiment of dielectrophoresis performed with use of the phoresis device 1 in accordance with the present embodiment, the inventors of the present invention conducted a study concerning the frequency of the AC voltage used in dielectrophoresis. That is, by applying the AC voltage to an experimental circuit on which a striped pattern of ITO was arranged, the inventors of the present invention checked how the frequency of the AC voltage is related to the movement speed (i.e., the phoresis speed) of the target object.

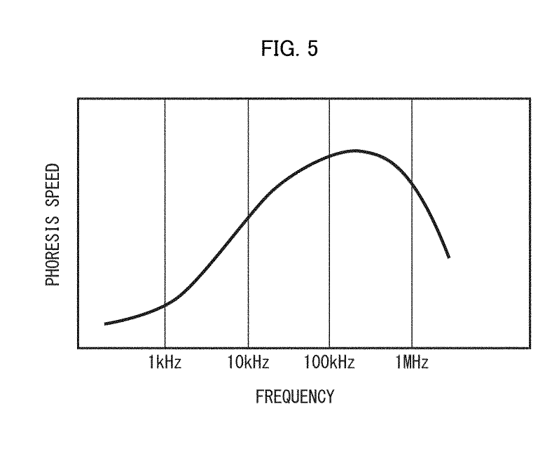

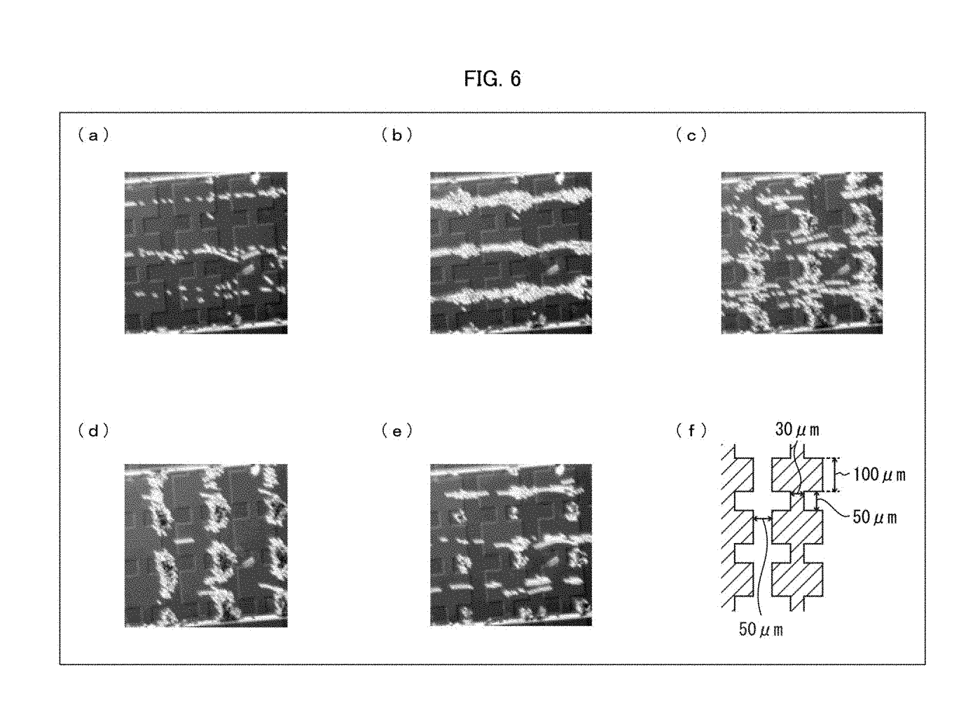

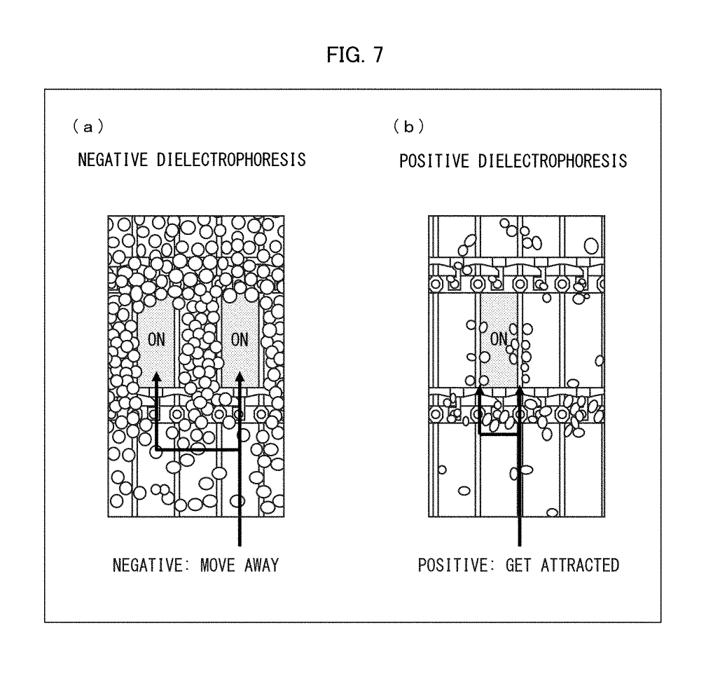

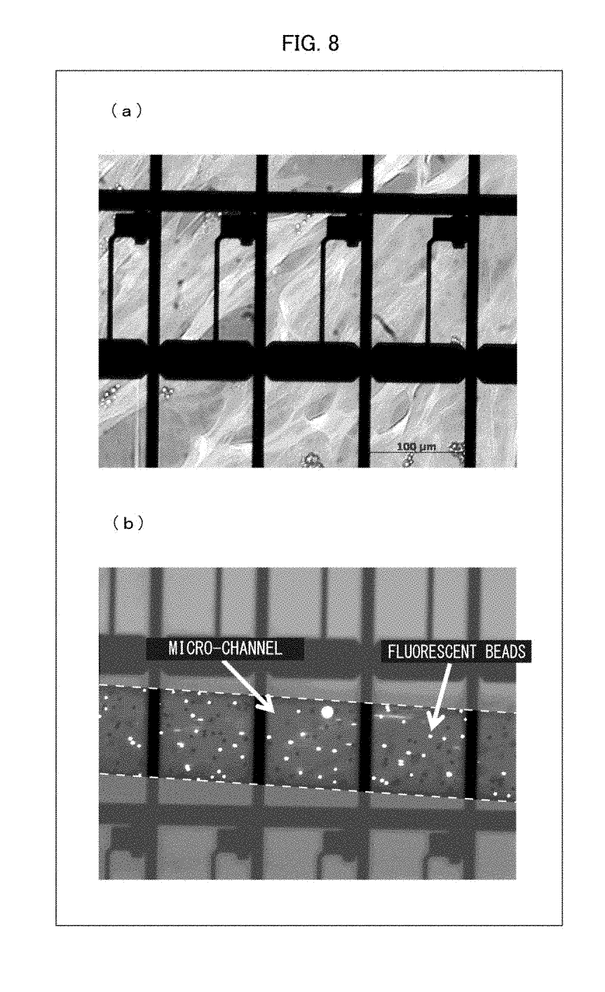

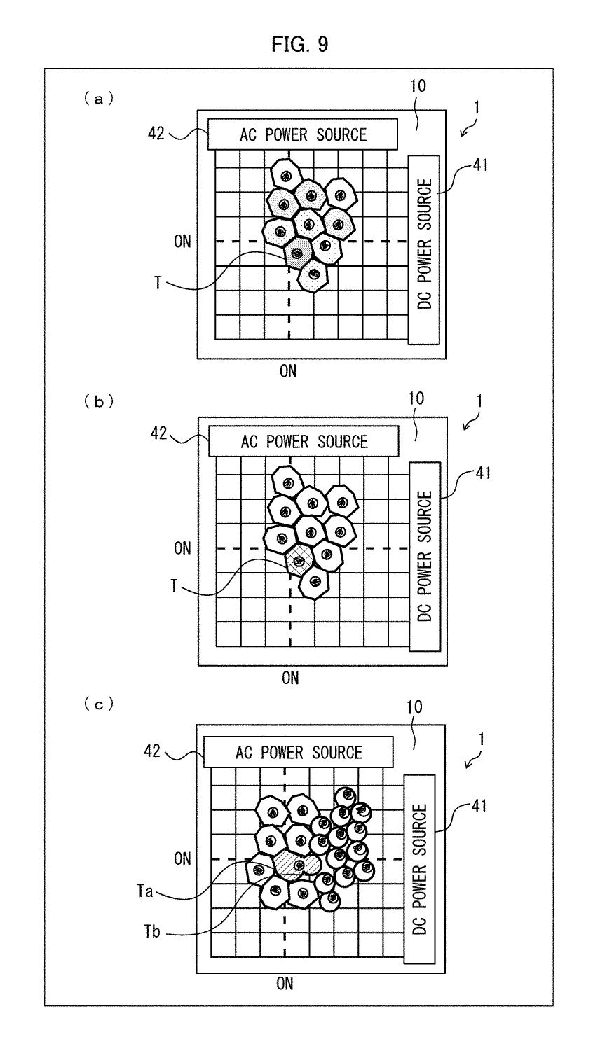

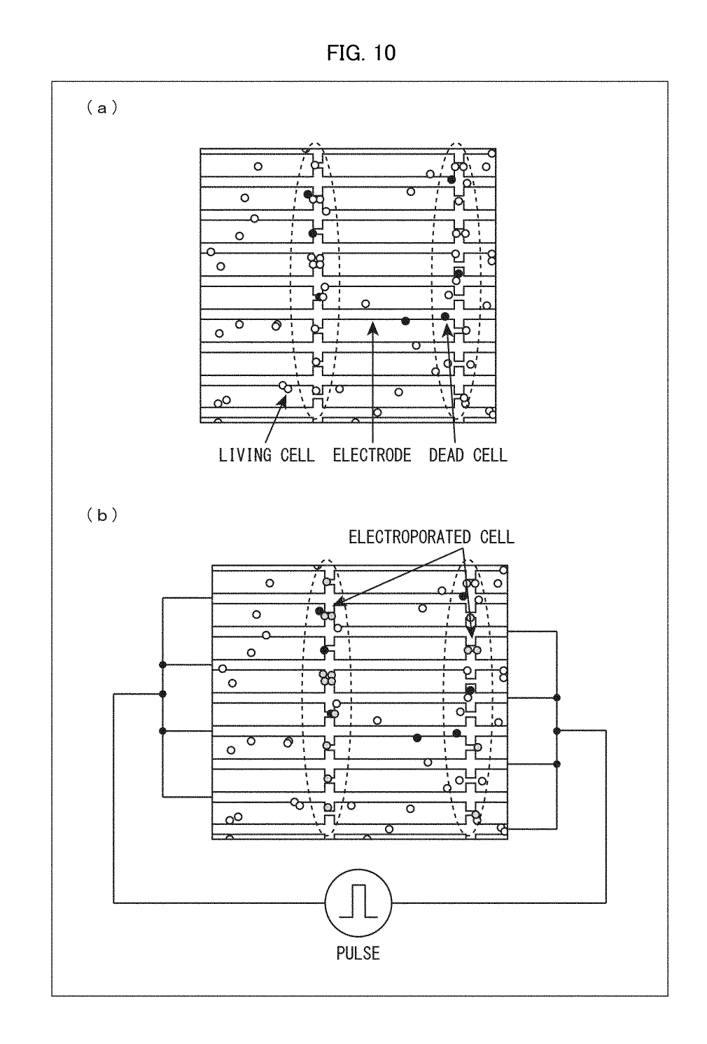

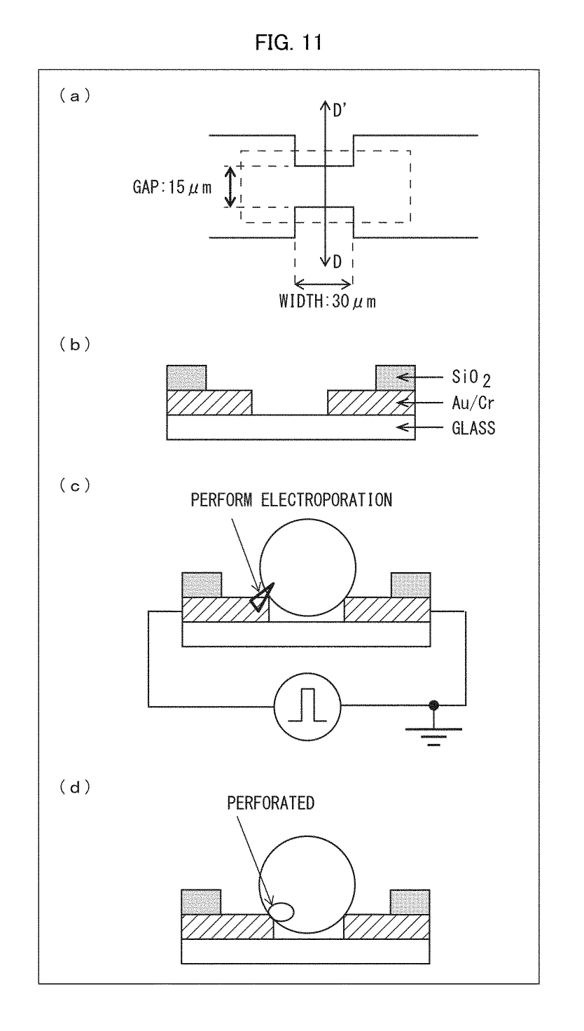

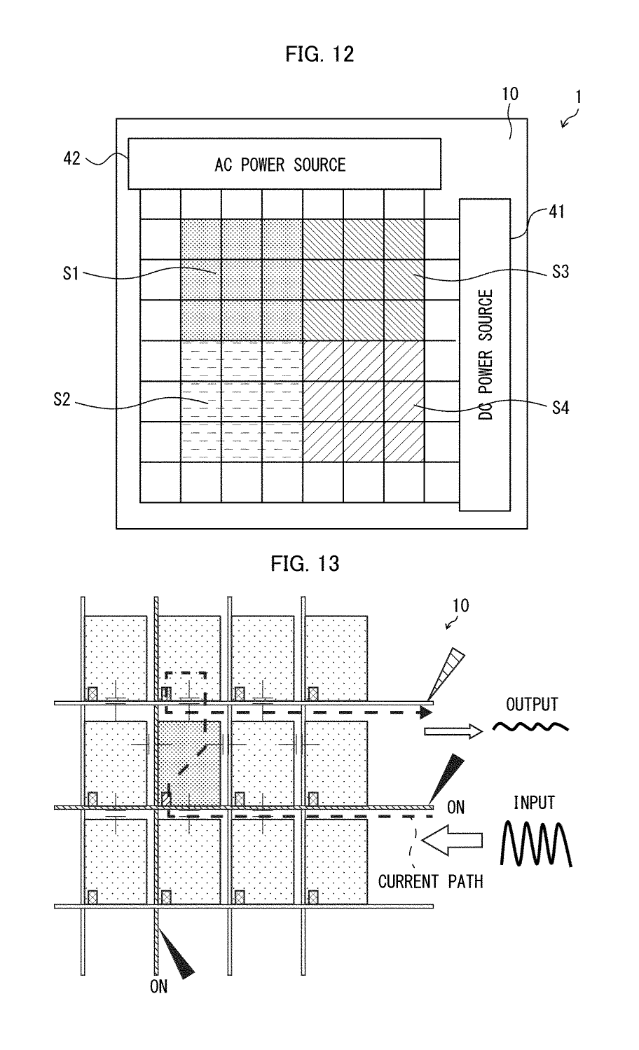

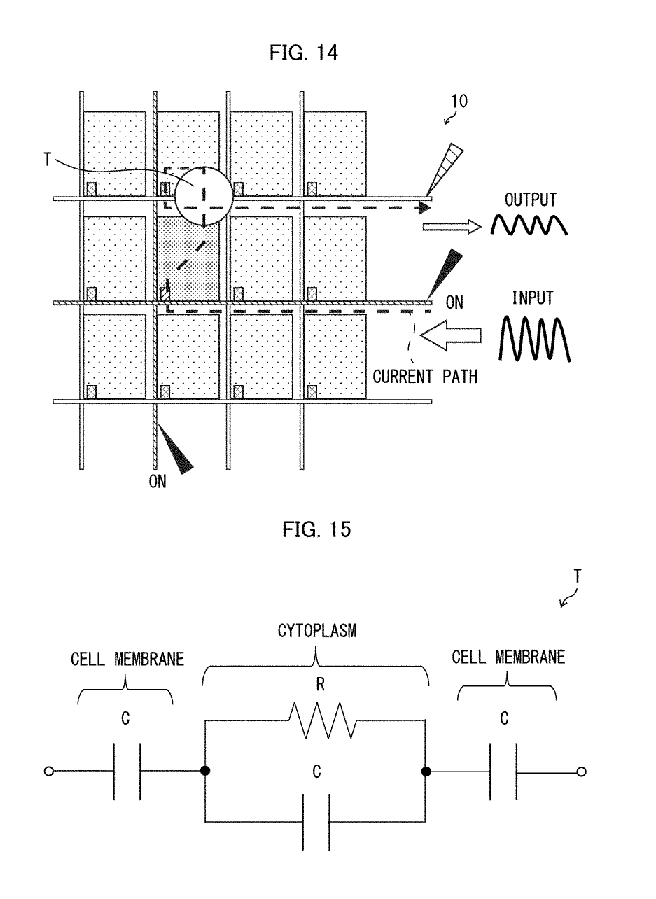

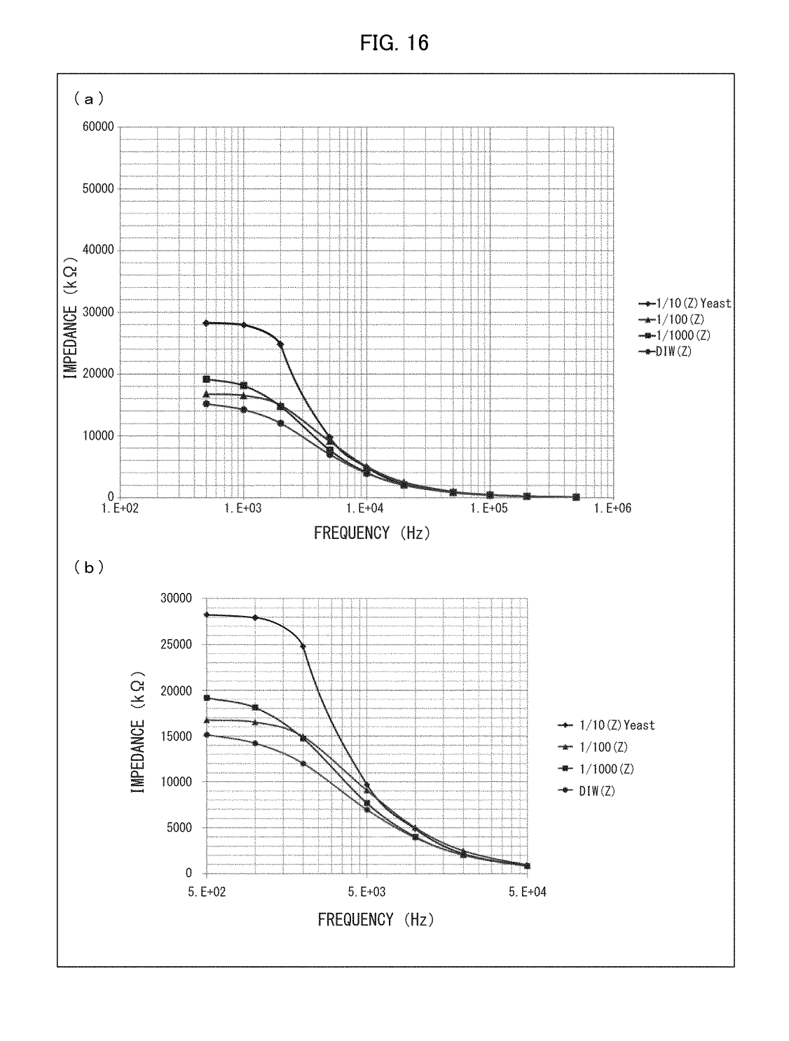

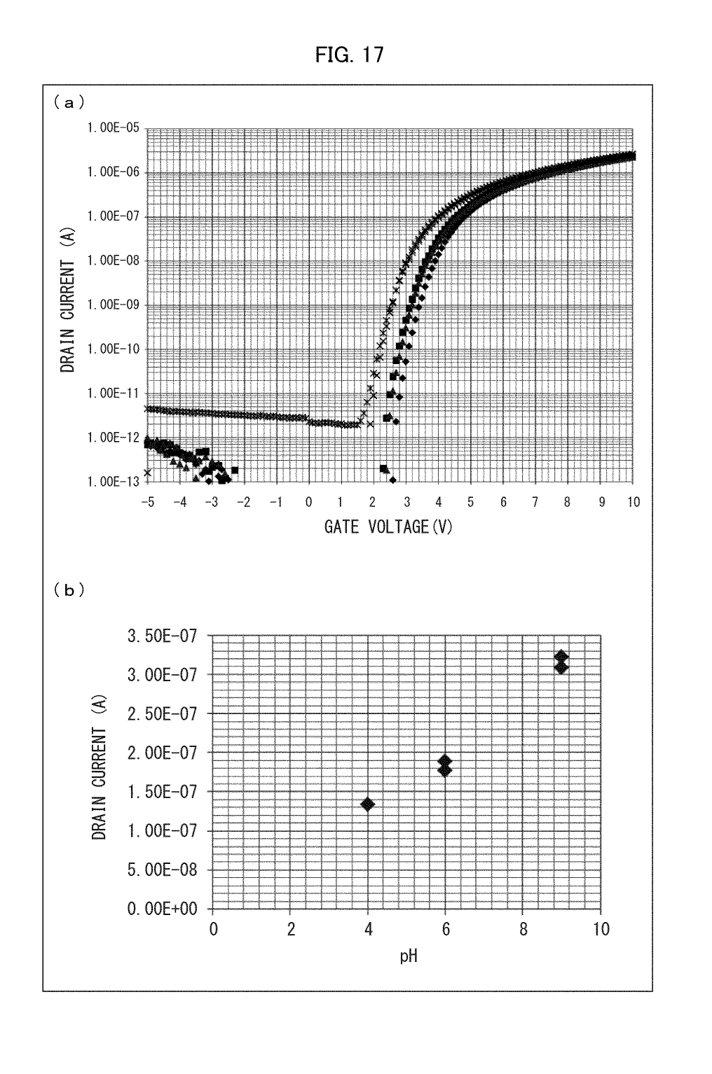

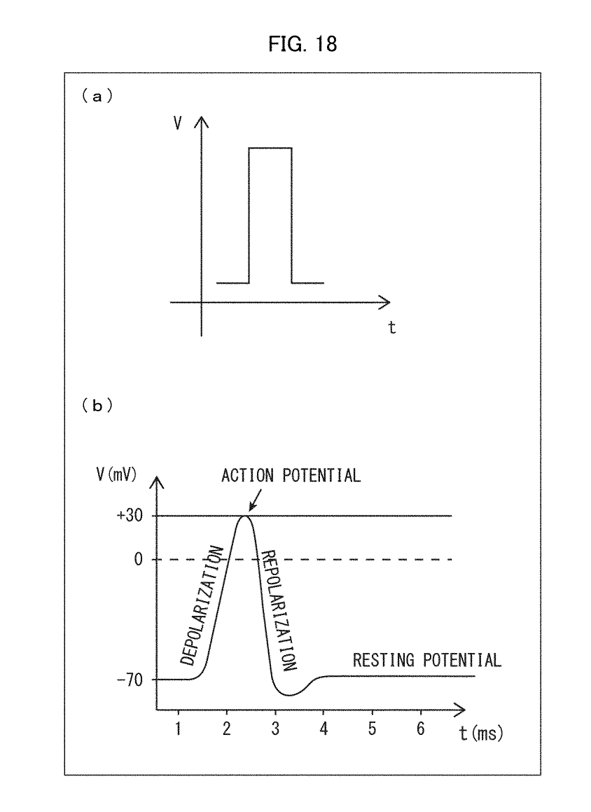

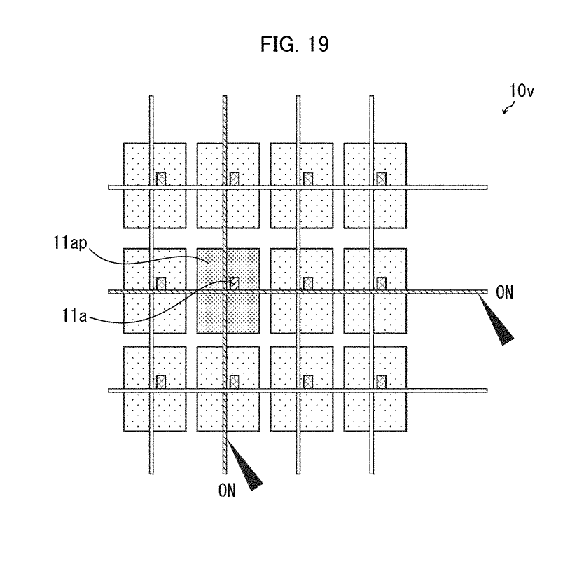

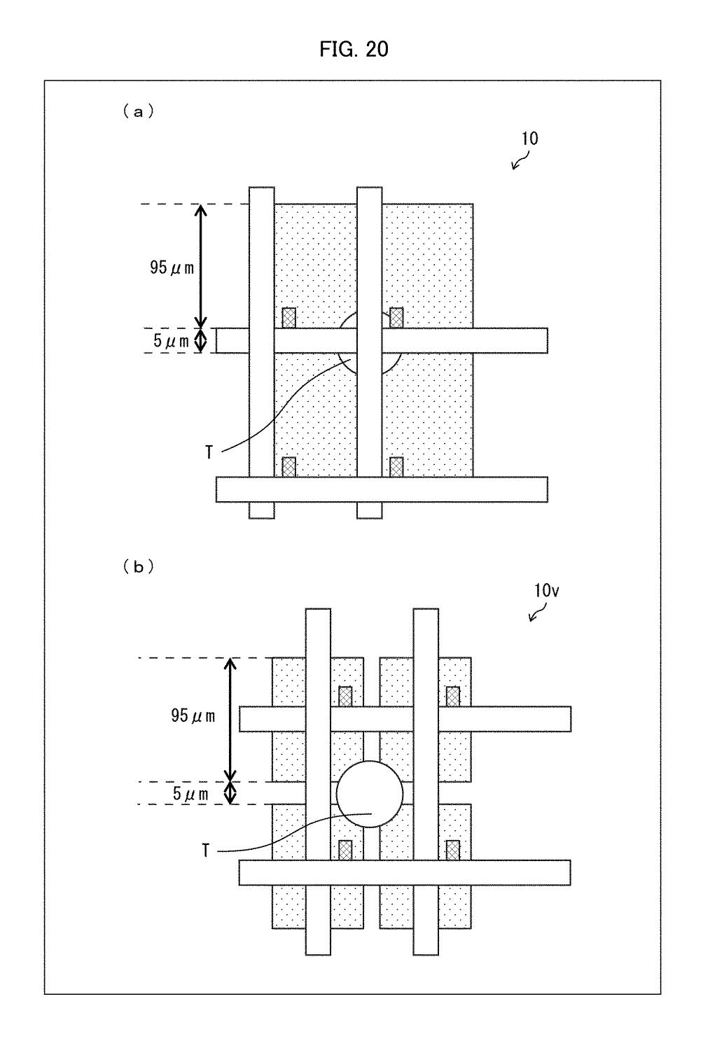

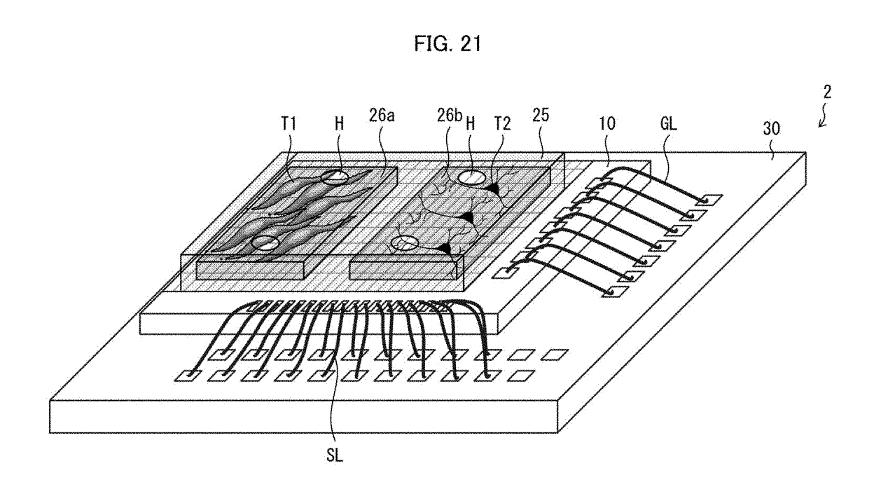

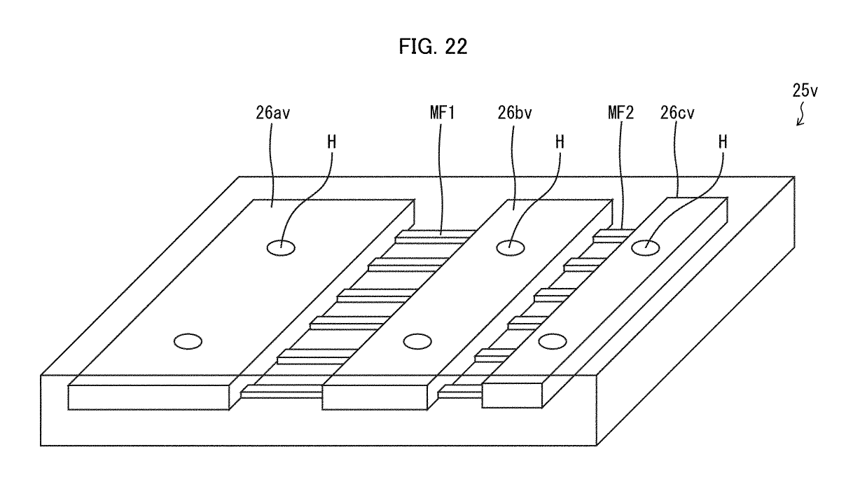

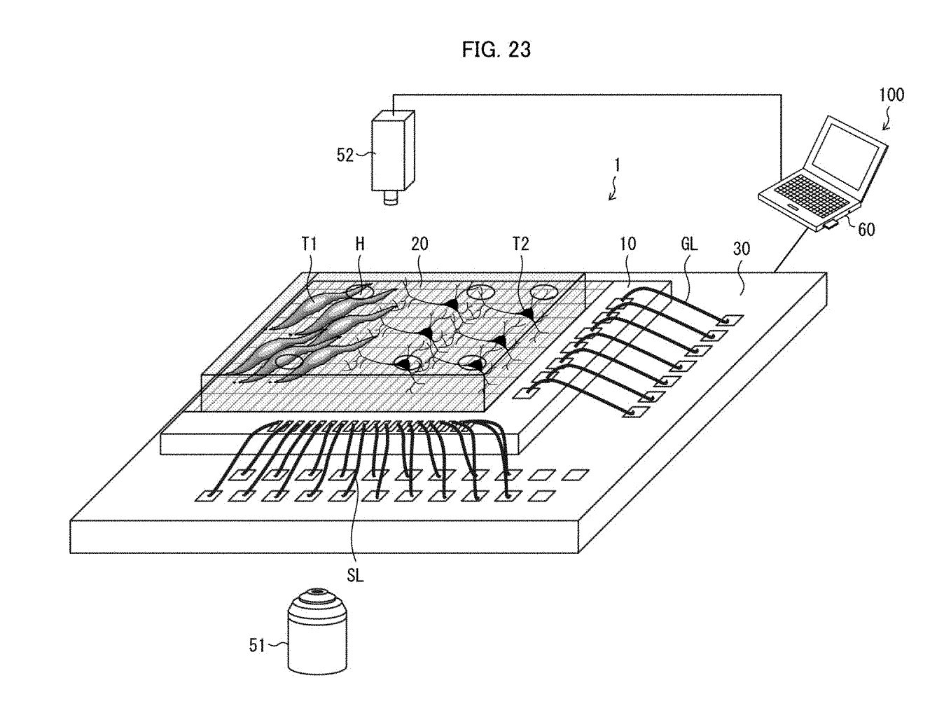

FIG. 5 is a graph, obtained by the above experiment, which indicates an experimental result of the relationship between the frequency of the AC voltage used for dielectrophoresis and the phoresis speed. Note that in the above experiment, micro-beads served as the target object of dielectrophoresis, and a sinusoidal voltage having a voltage (peak value) of 10 V was applied as the AC voltage.

As is clear from FIG. 5, it was confirmed that, in a case where micro-beads serve as the target object of dielectrophoresis, a relatively high phoresis speed is achieved when the frequency of the AC voltage is approximately 100 kHz to 500 kHz. Furthermore, it was confirmed that a relatively high phoresis speed is achieved when the frequency of the AC voltage is approximately 500 kHz.

The graph of FIG. 5 demonstrates that the phoresis speed significantly decreases in a case where the frequency of the AC voltage is higher than approximately 1 MHz. This is because, in a case where the frequency is relatively high, the dielectric loss at the target object becomes significant, and accordingly, it is not possible to effectively apply an electric field to the target object.

The graph of FIG. 5 also demonstrates that the phoresis speed significantly decreases in a case where the frequency of the AC voltage is lower than approximately 10 kHz. This is because, in a case where the frequency is relatively low, it is not possible to cause sufficient dielectric polarization to occur on the target object.

Note that a force FDEP, which is applied to the target object during dielectrophoresis, is known to be expressed by the following Equation (1):

$$\begin{matrix}[ Equation 1 ] & \\ 〈 {F}_{DEP} 〉 = 2 π {r}^{3} {ɛ}_{m} Re \{ \frac{{ɛ}_{p}^{*} - {ɛ}_{m}^{*}}{{ɛ}_{p}^{*} + 2 {ɛ}_{m}^{*}} \} ∇ {\vert {\vec{E}}_{rms} \vert}^{2} & ( 1 )\end{matrix}$$

In Equation (1), r denotes the radius of the target object, εp denotes the dielectric constant of the target object, em denotes the dielectric constant of the liquid that contains the target object, Erms denotes the rms value of the electric field applied to the target object, * (asterisk) is a symbol representing a complex number, and Re is a symbol representing the real part of the complex number.

From Equation (1), it is understood that the force FDEP applied to the target object also depends on the frequency of the electric field applied to the target object. The graph of FIG. 5 can be regarded as showing an example of the above dependency.

(a) through (e) of FIG. 6 each show how dielectrophoresis occurs at each frequency in a case where the frequency of the AC voltage is changed in the experiment of FIG. 5. Specifically, (a) of FIG. 6 shows how dielectrophoresis occurs in a case where the frequency is 1 kHz, (b) of FIG. 6 shows how dielectrophoresis occurs in a case where the frequency is 10 kHz, (c) of FIG. 6 shows how dielectrophoresis occurs in a case where the frequency is 100 kHz, (d) of FIG. 6 shows how dielectrophoresis occurs in a case where the frequency is 500 kHz, and (e) of FIG. 6 shows how dielectrophoresis occurs in a case where the frequency is 1 MHz. (f) of FIG. 6 is a schematic view of the striped pattern of ITO on the experimental circuit.

As is clear from (a) through (e) of FIG. 6, it was confirmed that, in a case where micro-beads serve as the target object, dielectrophoresis becomes particularly effective when the frequency is 500 kHz (in the case of (d) of FIG. 6).

However, as is clear from Equation (1), the frequency at which dielectrophoresis becomes particularly effective depends on the dielectric constant ep of the target object and the dielectric constant em of the liquid (in other words, the frequency depends on the kinds of the target object and of the liquid). As such, in a case where the target object is something (e.g., a cell) other than micro-beads, dielectrophoresis may become particularly effective at a frequency other than 500 kHz.

In view of the above, considering the margin of a settable frequency range and the like, the inventors of the present invention set, to 100 kHz, the frequency of the AC voltage used to perform dielectrophoresis in the phoresis device 1. Note, however, that a frequency other than 100 kHz can be used to perform dielectrophoresis in the phoresis device 1, depending on the kind of the target object and the like.

In a case where a cell serves as the target object, an excessively high peak value of the AC voltage may adversely affect the cell. The inventors of the present invention then set, to approximately 4 V, the peak value of the AC voltage used to perform dielectrophoresis in the phoresis device 1.

(Experimental Examples of Dielectrophoresis Performed with Use of Phoresis Device 1)

Next, experimental examples of dielectrophoresis performed in the phoresis device 1 will be described below with reference to FIG. 7. (a) and (b) of FIG. 7 each show an experimental example of dielectrophoresis performed in the phoresis device 1. The arrow shown in each of (a) and (b) of FIG. 7 indicates a pixel electrode(s) switched to the ON-state.

(a) of FIG. 7 shows an experimental example of dielectrophoresis performed in a case where micro-beads existing in the liquid injected in the chamber 20 served as the target object. In the case of (a) of FIG. 7, in order to switch two pixel electrodes of the TFT layer to the ON-state, (i) a DC voltage of 3 V was applied to each of the gate electrodes of the TFTs corresponding to the respective two pixel electrodes and (ii) a sinusoidal voltage having a voltage (peak value) of 3 V and a frequency of 100 kHz was applied to each of the source electrodes of the TFTs corresponding to the respective two pixel electrodes.

As is clear from (a) of FIG. 7, it was confirmed that, in a case where micro-beads serve as the target object, the micro-beads move so as to be away from the pixel electrodes that have been switched to the ON-state (in other words, so as to be away from the pixel electrodes that are higher in electrical potential than other surrounding pixel electrodes).

That is, it was confirmed that, on the TFT layer on which a non-uniform electric field has been formed, the micro-beads move from a position with high electrical potential towards a position with low electrical potential. As such, it was confirmed that negative dielectrophoresis occurs in a case where micro-beads serve as the target object.

(b) of FIG. 7 shows an experimental example of dielectrophoresis performed in a case where yeast cells existing in the liquid injected in the chamber 20 served as the target object. In the case of (b) of FIG. 7, in order to switch a single pixel electrode of the TFT layer to the ON-state, (i) a DC voltage of 1 V was applied to the gate electrode of the TFT corresponding to the single pixel electrode and (ii) a sinusoidal voltage having a voltage (peak value) of 4 V and a frequency of 100 kHz was applied to the source electrode of the TFT corresponding to the single pixel electrode.

As is clear from (b) of FIG. 7, it was confirmed that, in a case where yeast cells serve as the target object, the yeast cells move so as to be attracted to the pixel electrode that has been switched to the ON-state. That is, it was confirmed that, on the TFT layer on which a non-uniform electric field has been formed, the yeast cells move from a position with low electrical potential towards a position with high electrical potential. As such, it was confirmed that positive dielectrophoresis occurs in a case where yeast cells serve as the target object.

As described above, the inventors of the present invention newly conceived the technical idea of performing dielectrophoresis of the target object with use of the TFT substrate 10, and consequently realized the phoresis device 1. Note that, from Equation (1) provided above, it is understood that the polarity of dielectrophoresis depends on the dielectric constant εp of the target object and the dielectric constant εm of the liquid.

(Examples of Result of Optical Observation in Phoresis Device 1)

According to the phoresis device 1 in accordance with the present embodiment, the TFT substrate 10 has a light-transmitting region. It is therefore possible to move the target object by dielectrophoresis as well as to conduct optical observation of the target object. For example, the observer can observe, with the naked eyes or with use of an optical microscope, movement of the target object that is moved by dielectrophoresis.

By producing the chamber 20 as a transparent member, it is possible to conduct optical measurement of the target object even in a case where the phoresis device 1 includes the chamber 20.

FIG. 8 illustrates results of optical observation of the target object in the phoresis device 1. (a) of FIG. 8 illustrates a result of optical observation conducted in a case where epithelial cells existing in the liquid injected in the chamber 20 served as the target object. In the case of (a) of FIG. 8, a fluorescent marker was added to the liquid so as to facilitate the observation of the target object.

(b) of FIG. 8 illustrates a result of optical observation conducted in a case where micro-beads existing in the liquid injected in the chamber 20 served as the target object. Also in the case of (b) of FIG. 8, a fluorescent marker was added to the liquid so as to facilitate the observation of the target object.

The observation results illustrated in (a) and (b) of FIG. 8 are each an image obtained through observation under a microscope (e.g., a microscope 51 shown in FIG. 23 described later). Note that the target object such as the epithelial cells can be cultured on the TFT substrate 10 after dielectrophoresis.

The phoresis device 1 makes it possible to perform dielectrophoresis of the target object and to allow the target object to be subjected to optical measurement. Therefore, with the phoresis device 1, the observer can easily check whether the result of optical measurement of the target object is consistent with the result of electrical measurement of the target object. Moreover, the observer can also adjust, as appropriate, the condition of electrical measurement with reference to the result of optical measurement.

(Examples of Certain Manipulation of Target Object)

The phoresis device 1 can also form a second electric field, which differs from the electric field that causes dielectrophoresis by which the target object is moved, by (i) adjusting the voltage to be applied to the gate electrode of a TFT (i.e., the voltage to be generated by the DC power source 41) and (ii) adjusting the voltage to be applied to the source electrode of the TFT (i.e., the voltage to be generated by the AC power source 42). The second electric field corresponds to a certain manipulation of the target object other than dielectrophoresis.

Examples of the manipulation of the target object other than dielectrophoresis will be described below with reference to FIG. 9. (a) through (c) of FIG. 9 each illustrate a manipulation of the target object, other than dielectrophoresis, which is performed in the phoresis device in accordance with Embodiment 1 of the present invention.

Note that the manipulations shown in (a) through (c) of FIG. 9 each can be performed by causing the AC power source 42 to generate a certain pulsed voltage waveform. The manipulations shown in (a) through (c) of FIG. 9 can be switched by changing, for example, the amplitude, offset value, frequency, and/or duty ratio of the certain pulsed voltage waveform.

For simplification, the gate lines GL and the source lines SL are not shown in (a) through (c) of FIG. 9. (a) through (c) of FIG. 9 illustrate a case where cells T, Ta, and Tb which serve as the target object are placed directly on the TFT substrate 10.

(a) of FIG. 9 illustrates a case where the certain manipulation is a manipulation of performing electrical stimulation (electro-stimulation) of the cell T. The manipulation of performing electrical stimulation of the cell T can be performed for the purpose of activating the cell T.

(b) of FIG. 9 illustrates a case where the certain manipulation is a manipulation of performing electroporation on the cell T. The manipulation of performing electroporation of the cell T can be performed as a pretreatment performed prior to cell fusion shown in (c) of FIG. 9 described below.

(c) of FIG. 9 illustrates a case where the certain manipulation is a manipulation of performing cell fusion of the cells Ta and Tb (i.e., a plurality of cells). The manipulation of performing cell fusion of a plurality of cells can be performed for the purpose of conducting an experiment for the observation of a fused cell in its evolution or behavior.

(Experimental Example of Electroporation)

The inventors of the present invention performed electroporation on myeloma cells as an experimental example. The experimental example will be described below with reference to FIGS. 10 and 11.

(a) of FIG. 10 shows a state where the myeloma cells have not been electroporated in the phoresis device 1 yet. In the present experiment, fluorescent markers were added for determining whether the myeloma cells were alive or dead. Living cells were colored green whereas dead cells were colored red. For convenience, in (a) of FIG. 10, living cells are indicated in white, and dead cells are indicated in black.

In the present experiment, a fluorescein diacetate (FDA) was used as the fluorescent marker for distinguishing living cells, and a propidium iodide (PI) was used as the fluorescent marker for distinguishing dead cells.

(b) of FIG. 10 shows a state where the myeloma cells have been electroporated in the phoresis device 1. In the present experiment, electroporation was performed by applying, between electrodes (electrode patterns), a pulse having an amplitude of 4 V, an ON-time of 500 μs, and a duty ratio of 50% twice.

In a case where the cell membrane of a living cell is perforated with a hole by electroporation, the PI enters a myeloma cell (i.e., the living cell) via the hole. Note that the PI cannot enter a living cell having no hole. In the myeloma cell which has been electroporated, the fluorescent marker reacts with the core of the myeloma cell. As a result, the myeloma cell is colored pale orange that is a mixed color of green and red.

As such, in the present experiment, it was possible to identify cells colored pale orange as electroporated myeloma cells. For convenience, in (b) of FIG. 10, the electroporated myeloma cells are indicated in gray.

Meanwhile, another experiment was conducted as in the above experiment except that the amplitude of the pulse was 6 V. In this experiment, all myeloma cells were confirmed to be colored red. That is, it was confirmed that the myeloma cells completely die in a case where the amplitude of the pulse is 6 V.

(a) of FIG. 11 is an enlarged view of the electrode shown in FIG. 10. (b) of FIG. 11 is a cross-sectional view taken along the line D-D′ shown in (a) of FIG. 11. As shown in (b) of FIG. 11, the electrode is covered with a SiO₂ layer, except for an electrode portion (Au/Cr layer) at which electroporation is performed. It is therefore possible to perform electroporation only at the electrode portion that is not covered with the SiO₂ layer.

(c) of FIG. 11 is a schematic view showing how the cell placed on the electrode portion is electroporated by a pulse being applied to the electrode portion. (d) of FIG. 11 is a schematic view showing how the cell membrane is perforated by electroporation.

(Case where TFT Substrate 10 Serves as Sensor)

According to the phoresis device 1 in accordance with the present embodiment, it is possible to cause at least some portions of the TFT substrate 10 to serve as various sensors. FIG. 12 illustrates a case where some portions of the TFT substrate 10 serve as various sensors. For simplification, the gate lines GL and the source lines SL are not shown in FIG. 12.

In the example shown in FIG. 12, at least some portions of the TFT substrate 10 serve as an ion sensitive field effect transistor (ISFET) sensor S1, a resistive sensor S2, an electrostatic capacitive sensor S3, and an impedance sensor S4.

The ISFET sensor S1 serves to detect ion sensitive electrical potential. By causing at least part of the TFT substrate 10 to serve as the ISFET sensor, it is possible to detect swaps of a certain kind of ions (e.g., calcium ions, sodium ions, or potassium ions) in the target object. This makes it possible to check the activity level (life and death) of the target object.

The resistive sensor S2 serves to detect electrical resistance (R). By causing at least part of the TFT substrate 10 to serve as the resistive sensor, it is possible to detect whether the target object is present or absent at the position of the at least part of the TFT substrate 10.

The electrostatic capacitive sensor S3 serves to detect electrostatic capacitance (C). By causing at least part of the TFT substrate 10 to serve as the electrostatic capacitive sensor, it is possible to detect whether the target object is present or absent at the position of the at least part of the TFT substrate 10.

The impedance sensor S4 serves to detect impedance (Z). By causing at least part of the TFT substrate 10 to serve as the impedance sensor, it is possible to detect whether the target object is present or absent at the position of the at least part of the TFT substrate 10, as well as to check the activity level of the target object.

As such, by causing certain portions of the TFT substrate 10 to serve as various sensors, it is possible to detect, in the certain portions of the TFT substrate 10, (i) whether the target object is present or absent and (ii) the condition of the target object. It is therefore possible to detect, at an arbitrary position on the TFT substrate 10, (i) whether the target object is present or absent and (ii) the condition of the target object.

(Impedance Sensor)

Next, the fundamental concept of the impedance sensor in accordance with the present embodiment will be described below with reference to FIGS. 13 through 15.

FIG. 13 illustrates an example of a current path in a case where no cell is placed on the TFT substrate 10. FIG. 13 assumes a case where a single pixel electrode is in the ON-state. As shown in FIG. 13, a certain AC voltage (sinusoidal voltage) is applied to the source wiring of the TFT that corresponds to the pixel electrode in the ON-state. Accordingly, an alternating current flows in the TFT from the AC power source 42.

Here, electrostatic capacitance is formed between pixel electrodes of the TFT substrate 10. The alternating current therefore flows, via the electrostatic capacitance formed between the pixel electrode in the ON-state and an adjacent pixel electrode which is adjacent to the pixel electrode in the ON-state, into the TFT that corresponds to the adjacent pixel electrode. The alternating current is then outputted from the source wiring that corresponds to the adjacent pixel electrode.

Note that, in the TFT that corresponds to a pixel electrode in the OFF-state, the electric current flowing from another pixel electrode in the OFF-state is ignorable because the resistance between the source and the drain is extremely large.

Therefore, in the TFT substrate 10, a measurement system (circuit network) is formed in which (i) an AC voltage V*, which is to be applied to the source wiring of the TFT that corresponds to a pixel electrode in the ON-state, is regarded as an input signal and (ii) an alternating current I*, which is to be outputted from the source wiring that corresponds to the TFT of an adjacent pixel electrode which is adjacent to the pixel electrode in the ON-state, is regarded as an output signal. Here, * (asterisk) is a symbol representing a complex number.

Impedance Z of the measurement system is expressed as Z=V*/I*. Since the AC voltage V* is known, it is possible to calculate the impedance Z by making an arrangement such that the alternating current I* is detectable in at least part of the TFT substrate 10. In other words, it is possible to cause the at least part of the TFT substrate 10 to serve as an impedance sensor. The impedance Z can be measured by use of a device such as an LCR meter.

Moreover, by arranging the measurement system so that only the real part of the impedance Z is measurable, it is possible to cause at least part of the TFT substrate 10 to serve as a resistive sensor. Meanwhile, by arranging the measurement system so that only the imaginary part of the impedance Z is measurable, it is possible to cause at least part of the TFT substrate 10 to serve as an electrostatic capacitive sensor.

FIG. 14 illustrates an example of a current path in a case where a cell T is placed on the TFT substrate 10. FIG. 14 assumes a case where the cell T is placed between the pixel electrode in the ON-state and an adjacent pixel electrode which is adjacent to the pixel electrode in the ON-state.

FIG. 15 shows the electrical equivalent circuit of the cell T. FIG. 15 assumes a model in which the cell T includes a cell membrane and cytoplasm. The cell membrane can be, as shown in FIG. 15, regarded as electrostatic capacitance. This approximation is particularly effective in a high frequency region.

In contrast, because the cytoplasm includes an electrolyte, the cytoplasm can be regarded as having not only electrostatic capacitance but also electrical resistance. Accordingly, the cytoplasm is expressed as an RC parallel circuit. Therefore, it can be understood that the cell T has certain impedance that is formed by the circuit shown in FIG. 15.

As such, in a case where the cell T is placed as shown in FIG. 14, the alternating current I* is outputted, mainly via the cell T, from the source wiring that corresponds to the TFT of the adjacent pixel electrode, which is adjacent to the pixel electrode in the ON-state.

Accordingly, the alternating current I* in the case of FIG. 14 differs in magnitude from that in the case of FIG. 13. In other words, the impedance Z of the measurement system in the case of FIG. 14 differs from that of the measurement system in the case of FIG. 13. Therefore, by measuring the impedance Z, it is possible to detect whether the cell T is present in the measurement system.

Note that, in a case where at least part of the TFT substrate serves as an impedance sensor, the pixel electrodes in the region of the at least part of the TFT substrate are sequentially switched to the ON-state. By thus conducting measurement of the region where the cell T is placed, it is possible to map the position and the condition of the cell T.

FIG. 16 is a graph showing an example of a result of an experiment conducted in the case where the TFT substrate 10 served as an impedance sensor. In this experiment, impedance measurement was conducted on four kinds of liquids, namely, (i) a 10 percent diluted yeast solution (legend: 1/10(Z)Yeast), (ii) a 1 percent diluted yeast solution (legend: 1/100(Z)), (iii) a 0.1 percent diluted yeast solution (legend: 1/1000(Z)), and (iv) pure water (legend: DIW(Z)).

(a) of FIG. 16 is a graph showing the result of impedance measurement made on each liquid under the conditions in which the frequencies of the AC voltage were varied in the range from 500 Hz to 500 kHz. (a) of FIG. 16 demonstrates a tendency that the impedance increases with increase in concentration of yeast cells contained in the liquid.

(b) of FIG. 16 is an enlarged view of the frequency range from 500 Hz to 50 kHz shown in the graph of (a) of FIG. 16. (b) of FIG. 16 demonstrates that the above tendency, in which the impedance increases with increase in concentration of yeast cells contained in the liquid, is particularly significant in a relatively low frequency region from approximately 500 Hz to 10 kHz.

As such, in a case where at least part of the TFT substrate 10 serves as an impedance sensor, a frequency suitable for measurement is set as appropriate depending on the kind of object subject to impedance measurement.

(Isfet Sensor)

FIG. 17 is a graph showing an example of a result of an experiment conducted in a case where the TFT substrate 10 served as an impedance sensor. In the present embodiment, the TFT serves as an ISFET sensor by measuring the drain current of a TFT.

In this experiment, the ISFET sensor was used as a pH sensor, and pH measurement was conducted on three kinds of liquids, namely, (i) a borate pH standard solution (pH=9), (ii) pure water (pH=7), and (iii) a phthalate pH standard solution (pH=4).

(a) of FIG. 17 is a graph showing the result of drain current measurement made under the conditions in which the drain voltage of a TFT was 0.5 V and the gate voltage of the TFT was varied in a range from −5 V to 10 V. (a) of FIG. 17 demonstrates that a measured value of the drain current varies depending on a pH value of a liquid even when the value of the gate voltage is fixed.

Therefore, if the correspondence between the gate voltage and the pH is known, the pH of a liquid can be calculated from a measured drain current. In the present experiment, the correspondence between the gate voltage and the pH was expressed, by using the experimental data obtained in advance, as a polynomial “y=0.0157x²+0.0717x+7.664” where x denotes the pH and y denotes the gate voltage.

(b) of FIG. 17 is a graph showing the relationship between a measured drain current and a calculated pH. According to (b) of FIG. 17, three values, i.e., pH=9, pH=6, and pH=4, are obtained as the pH.

That is, an appropriate pH is calculated for each of the borate pH standard solution and the phthalate pH standard solution. Note that, because pure water absorbs CO₂, the pH of pure water decreases during the experiment. As a result, the pH of pure water is calculated to be a value lower than pH=7.

(Examples of Electrical Stimulation)

As described earlier, the AC power source 42 can generate a pulsed voltage waveform, and the simulating circuit 43 can generate a voltage signal that serves as simulated stimulation. Electrical stimulation will be described below with reference to FIG. 18.

(a) of FIG. 18 shows the pulsed voltage waveform generated by the AC power source 42. The electrical stimulation caused by the pulsed voltage waveform is also referred to as pulse stimulation. The pulse stimulation can be used for the purpose of, for example, giving electrical stimulation to a nerve cell T1.

(b) of FIG. 18 shows the voltage waveform of simulated stimulation generated by the simulating circuit 43. The simulated stimulation refers to a voltage waveform that simulates time-varying changes of the membrane potential of the nerve cell T2. In the voltage waveform shown in (b) of FIG. 18, depolarization, repolarization, a resting potential, and an action potential are simulated.

It can be understood that the simulated stimulation is electrical stimulation that simulates the biological stimulation given to the nerve cell T2 more precisely than the pulse stimulation. Using the simulated stimulation enables a more precise experiment and less electrical stress to the nerve cell T1, in comparison with using the pulse stimulation.

The simulating circuit 43 can also generate a voltage waveform that simulates the membrane potential of the motor neuron connected to the muscle cell T1. Note that this motor neuron can be cultured on the TFT substrate 10.

(Effect of Phoresis Device 1)

As described above, according to the phoresis device 1 in accordance with the present embodiment, it is possible to realize, with use of the TFT substrate 10, a phoresis device for moving a target object (e.g., a cell) by dielectrophoresis. Moreover, by using the TFT substrate 10 having a light-transmitting region, it is possible to move the target object by dielectrophoresis as well as to allow the target object to be subjected to optical measurement.

Moreover, with the phoresis device 1 in accordance with the present embodiment, it is possible to form a second electric field, which differs from the electric field that causes dielectrophoresis by which the target object is moved, with use of the plurality of TFTs of the TFT substrate 10. By forming the second electric field, it is possible to perform a certain manipulation (e.g., electrical stimulation, electroporation, or cell fusion) of the target object other than dielectrophoresis.

Moreover, by causing at least some portions of the TFT substrate 10 to serve as various sensors (e.g., the ISFET sensor, the resistive sensor, the electrostatic capacitive sensor, and the impedance sensor), it is possible to detect, at the positions at which the various sensors are located, (i) whether the target object is present or absent and (ii) the condition of the target object.

### Modified Example

In a phoresis device in accordance with an embodiment of the present invention, the arrangement of the gate wirings and the source wirings on the TFT substrate is not limited to that of Embodiment 1. A modified example of the TFT substrate will be described below with reference to FIGS. 19 and 20.

FIG. 19 shows the configuration of a TFT substrate 10v, which is a modified example of the TFT substrate 10 in accordance with Embodiment 1. The TFT substrate 10v differs from the TFT substrate 10 in accordance with Embodiment 1 (see FIG. 3) in that the gate wirings and the source wirings are arranged so as not to overlap with the edges of the pixel electrodes (ITO electrodes).

Next, the advantage brought about by the TFT substrate 10v will be described below with reference to FIG. 20. (a) of FIG. 20 shows the positional relationship between the pixel electrodes and the electrode wirings on the TFT substrate 10 shown in FIG. 3. (b) of FIG. 20 shows the positional relationship between the pixel electrodes and the electrode wirings on the TFT substrate 10v shown in FIG. 19.

Note that FIG. 20 assumes a case where each pixel electrode has a size of 95 μm, the pixel pitch is 100 μm, each wiring has a width of 5 μm, the gate wirings and the source wirings are each made of an opaque metal material (e.g., Al), and the cell T has a size of the order of several micrometers to several tens of micrometers.

According to the TFT substrate 10, the opaque gate wirings and the opaque source wirings are arranged so as to overlap with the edges of the pixel electrodes (see (a) of FIG. 20). Therefore, in a case where the cell T is present between the pixel electrodes, the cell T is hidden by the opaque gate wirings and the opaque source wirings. In such a case, it is not possible to conduct optical observation of the cell T.

In contrast, according to the TFT substrate 10v, the opaque gate wirings and the opaque source wirings are arranged so as not to overlap with the edges of the pixel electrodes (see (b) of FIG. 20). It is therefore possible to conduct optical observation of the cell T even in a case where the cell T is present between the pixel electrodes, because the cell T is never hidden by the opaque gate wirings and the opaque source wirings.

The TFT substrate 10v therefore makes it possible to conduct optical observation of the cell T even in a case where an opaque metal material such as Al is used to reduce the electrical resistance of the gate wirings and the source wirings. The TFT substrate 10v is beneficial because, in a case where dielectrophoresis is performed, a relatively large number of target objects may move to the vicinities of boundaries between the pixel electrodes.

### Embodiment 2

The following description will discuss another embodiment of the present invention with reference to FIG. 21. For convenience, members having functions identical to those described in the foregoing embodiment are given the same reference numerals, and the descriptions of such members are omitted.

FIG. 21 shows the configuration of a phoresis device 2 in accordance with the present embodiment. The configuration of the phoresis device 2 in accordance with the present embodiment can be obtained by replacing, in the phoresis device 1 in accordance with Embodiment 1, the chamber 20 with a chamber 25 (restricting member). For simplification, the DC power source 41, the AC power source 42, and the simulating circuit 43 are not shown in FIG. 21.

According to the phoresis device 2, the chamber 25 includes a first chamber 26a (restricting member) and a second chamber 26b (restricting member). The first chamber 26a restricts the range of movement of a liquid containing a muscle cell T1. The second chamber 26b restricts the range of movement of a liquid containing a nerve cell T2.

The first chamber 26a has injection holes H via which a liquid is injected into the first chamber 26a from the outside of the chamber 25. Similarly, the second chamber 26b has injection holes H via which a liquid is injected into the second chamber 26b from the outside of the chamber 25.

The first chamber 26a and the second chamber 26b are spaced from each other so as not to communicate with each other. It is therefore possible to separate the liquids so that the liquid stored in the first chamber 26a is not mixed with the liquid stored in the second chamber 26b.

According to the phoresis device 2, by providing a plurality of chambers (the first chamber 26a and the second chamber 26b), it is possible to perform dielectrophoresis and a certain manipulation individually on the muscle cell T1 and the nerve cell T2, which are contained in the respective liquids thus separated from each other. This further improves the convenience of the phoresis device.

### Modified Example

Note that, even in a case where a phoresis device in accordance with an embodiment of the present invention includes a plurality of restricting members, the configuration of the plurality of restricting members is not limited to that of Embodiment 2. A modified example of the restricting member will be described blow with reference to FIG. 22.

FIG. 22 shows the configuration of a chamber 25v (restraining member), which is a modified example of the chamber 25 in accordance with Embodiment 2. FIG. 22 assumes a case where each member of the chamber 25v is made of PDMS and is thus transparent.

The chamber 25v has, as shown in FIG. 22, the shape of a substantially rectangular parallelepiped. The chamber 25v includes a first chamber 26av (restricting member), a second chamber 26bv (restricting member), and a third chamber 26cv (restricting member). As such, the number of restricting members can be three or more.

Each of the first chamber 26av, the second chamber 26bv, and the third chamber 26cv has injection holes H via which a liquid is injected thereinto from the outside of the chamber 25v.

Moreover, the chamber 25v includes (i) micro-flow paths MF1 which connect the first chamber 26av with the second chamber 26bv and (ii) micro-flow paths MF2 which connect the second chamber 26bv with the third chamber 26cv. Note that each of the micro-flow paths MF1 and MF2 can have a width of the order of several micrometers.

By providing the micro-flow paths MF1 and MF2, it is possible to connect the first chamber 26av, the second chamber 26bv, and the third chamber 26cv.

For example, the chamber 25v is suitably used to culture the cells contained in the liquids within the individual chambers and then mix the liquids within the chambers with each other. Furthermore, according to the chamber 25v, it is also possible to introduce axons of nerve cells from the chamber in which the nerve cells are stored (e.g., from the first chamber 26av) to the chamber in which muscle cells are stored (e.g., to the second chamber 26bv).

### Embodiment 3

The following description will discuss another embodiment of the present invention with reference to FIG. 23. For convenience, members having functions identical to those described in the foregoing embodiments are given the same reference numerals, and the descriptions of such members are omitted.

FIG. 23 shows the configuration of an observation system 100 in accordance with the present embodiment. The observation system 100 includes a phoresis device 1, a microscope 51, a digital camera 52, and a control device 60. For simplification, the DC power source 41, the AC power source 42, and the simulating circuit 43 are not shown in FIG. 23.

The microscope 51 is an optical observation device that serves to facilitate optical observation of a target object in the phoresis device 1. The microscope 51 can be manually operated by the observer. The microscope 51 can be, for example, an inverted microscope for observing the target object from the substrate 30 side, or an erected microscope for observing the target object from the chamber 20 side.

The microscope 51 is preferably an inverted microscope. In a case where the microscope 51 is an inverted microscope, it is possible to more easily observe the target object at a high magnification, because no chamber 20 exists between the microscope 51 and the target object.

The digital camera 52 is an optical observation device that serves to further facilitate the optical observation of the target object in the phoresis device 1. The operation of the digital camera 52 is controlled by the control device 60. Therefore, with the digital camera 52, it is possible to conduct optical observation of the target object (i.e., capture an image of the target object) without a need for the observer to make any manual operation.

The control device 60 is a member that comprehensively controls the operations of the digital camera 52 and the phoresis device 1. The control device 60 can be, for example, a personal computer (PC). The control device 60 is connected to the digital camera 52 and a substrate 30.

With the control device 60, it is therefore possible to control the operation of each member (e.g., the DC power source 41, the AC power source 42, and the like) of the phoresis device 1 via the substrate 30. This makes it possible for the user to easily perform dielectrophoresis and various manipulations of the target object via the control device 60.

(Object of Observation System 100)

The observation system 100 is intended to realize a platform that allows for in-vitro analysis for the researches that place importance on cell-to-cell interactions.

The observation system 100 can be used to conduct, for example, (i) investigation of disease processes or (ii) research on treatment for a disease such as ALS or myopathy.

According to the phoresis device 1, it is possible to give electrical stimulation only to a muscle cell or a nerve cell that has been arranged at a specific position on a TFT substrate 10. This makes it possible to culture a complete muscle unit (a muscle cell with a motor neuron).

With the observation system 100, it is also possible to treat more than two kinds of cells as experimental objects, so as to build a model that is more similar to a living body. For example, in order to facilitate the research on cell-to-cell interactions, it is possible to introduce a sensory receptor (e.g., a cell that senses heat), place the cell on the TFT substrate 10, and connect the cell with a nerve cell.

[Recap]

A phoresis device in accordance with a first aspect of the present invention is a phoresis device (1) for moving a target object (e.g., cell T) by dielectrophoresis, the phoresis device including: a thin film transistor substrate (TFT substrate 10) which supports the target object and which is configured to form an electric field that causes the dielectrophoresis, the thin film transistor substrate including a plurality of transistors (TFT 11a), the target object being moved through application of a voltage to part of the plurality of transistors of the thin film transistor substrate.

According to the above configuration, it is possible to realize, with use of a TFT substrate, a phoresis device for moving the target object by dielectrophoresis.

The phoresis device in accordance with a second aspect of the present invention is preferably configured such that, in the first aspect of the present invention, the thin film transistor substrate has a light-transmitting region.

According to the above configuration, by using the TFT substrate having a light-transmitting region, it is possible to move the target object by dielectrophoresis as well as to allow the target object to be subjected to optical measurement.

The phoresis device in accordance with a third aspect of the present invention is preferably configured to further include, in the first or second aspect of the present invention, a restricting member (chamber 20), arranged on the thin film transistor substrate, which restricts a range of movement of the target object.

According to the above configuration, the restricting member is provided on the TFT substrate. This allows the phoresis device to handle a liquid that contains the target object.

The phoresis device in accordance with a fourth aspect of the present invention is preferably configured such that, in the third aspect of the present invention, the restricting member is made of polydimethyl siloxane, epoxy resin, polymethyl methacrylate, polyvinylidene difluoride, glass, or quartz.

According to the above configuration, it is possible to produce the restricting member as a transparent member. This makes it possible to allow the target object to be subjected to optical measurement even in a case where the phoresis device includes the restricting member.

The phoresis device in accordance with a fifth aspect of the present invention is preferably configured such that, in any one of the first through fourth aspects of the present invention, at least part of the thin film transistor substrate serves as at least one of an ion sensitive field effect transistor (ISFET sensor S1), a resistive sensor (S2), an electrostatic capacitive sensor (S3), and an impedance sensor (S4).

According to the above configuration, the TFT substrate serves as the above sensors. This makes it possible to evaluate the presence/absence of the target object or the condition of the target object. For example, in a case where the target object is a cell, it is possible to determine the activity level (life or death) of the cell by measuring the impedance of the cell with use of the impedance sensor.

The phoresis device in accordance with a sixth aspect of the present invention is preferably configured such that, in any one of the first through fifth aspects of the present invention, the thin film transistor substrate is configured to form a second electric field, which differs from the electric field that causes the dielectrophoresis by which the target object is moved; and the second electric field corresponds to a certain manipulation of the target object other than the dielectrophoresis.

According to the above configuration, the second electric field is formed. This makes it possible to perform a certain manipulation of the target object other than dielectrophoresis.

The phoresis device in accordance with a seventh aspect of the present invention is preferably configured such that, in the sixth aspect of the present invention, the target object is a cell; and the certain manipulation is a manipulation of electrically stimulating the cell, a manipulation of electroporating the cell, or a manipulation of performing cell fusion of a plurality of the cells.

The above configuration makes it possible to perform a manipulation of electrically stimulating the cell, a manipulation of electroporating the cell, or a manipulation of performing cell fusion of a plurality of cells.

### Supplemental Notes

The present invention is not limited to the embodiments, but can be altered by a skilled person in the art within the scope of the claims. The present invention also encompasses, in its technical scope, any embodiment derived by combining technical means disclosed in differing embodiments. Further, it is possible to form a new technical feature by combining the technical means disclosed in the respective embodiments.

### REFERENCE SIGNS LIST

1, 2: Phoresis device
10: TFT substrate (thin film transistor substrate)
11a: TFT (transistor)
20, 25, 25v: Chamber (restricting member)
26a, 26av: First chamber (restricting member)
26b, 26bv: Second chamber (restricting member)
26cv: Third chamber (restricting member)
100: Observation system
51: Ion sensitive field effect transistor (ISFET sensor)
S2: Resistive sensor
S3: Electrostatic capacitive sensor
S4: Impedance sensor
T, Ta, Tb: Cell (target object)
T1: Muscle cell (target object)
T2: Nerve cell (target object)

1. A phoresis device for moving a target object by dielectrophoresis, the phoresis device comprising:
a thin film transistor substrate which supports the target object and which is configured to form an electric field that causes the dielectrophoresis, the thin film transistor substrate including a plurality of transistors,
the target object being moved through application of a voltage to part of the plurality of transistors of the thin film transistor substrate.

2. The phoresis device as set forth in claim 1, wherein:
the thin film transistor substrate has a light-transmitting region.

3. The phoresis device as set forth in claim 1, further comprising:
a restricting member, arranged on the thin film transistor substrate, which restricts a range of movement of the target object.

4. The phoresis device as set forth in claim 3, wherein:
the restricting member is made of polydimethyl siloxane, epoxy resin, polymethyl methacrylate, polyvinylidene difluoride, glass, or quartz.

5. The phoresis device as set forth in claim 1, wherein:
at least part of the thin film transistor substrate serves as at least one of an ion sensitive field effect transistor, a resistive sensor, an electrostatic capacitive sensor, and an impedance sensor.

6. The phoresis device as set forth in claim 1, wherein:
the thin film transistor substrate is configured to form a second electric field, which differs from the electric field that causes the dielectrophoresis by which the target object is moved; and
the second electric field corresponds to a certain manipulation of the target object other than the dielectrophoresis.

7. The phoresis device as set forth in claim 6, wherein:
the target object is a cell; and
the certain manipulation is a manipulation of electrically stimulating the cell, a manipulation of electroporating the cell, or a manipulation of performing cell fusion of a plurality of the cells.
